(19) United States
(12) Patent Application Publication
(10) Pub. No.: US 2007/0255200 A1
(43) Pub. Date: Nov. 1, 2007

(54) Time-indicating syringe-in-syringe mixing devices and related methods for storing and dispensing two-part dental compositions

(75) Inventors: Bruce S. McLean, Sandy, UT (US); Dan E. Fischer, Sandy, UT (US)

Correspondence Address: WORKMAN NYDEGGER, 60 EAST SOUTH TEMPLE, 1000 EAGLE GATE TOWER, SALT LAKE CITY, UT, 84111, US

(73) Assignee: Ultradent Products, Inc., South Jordan, UT (US)

(21) Appl. No.: 11/414,964

(22) Filed: May 1, 2006

(51) Int. Cl. A61M 37/00

(52) U.S. Cl. 604/82

(57) ABSTRACT

A time-indicating syringe-in-syringe mixing system for mixing, storing and dispensing a two-part composition that, when mixed together, yields a time-sensitive dental composition. The system includes a first plunger, a hollow second plunger configured to contain a first component, a syringe barrel configured to contain a second component, structure for recording a date when a first component was mixed with a second component, and structure for protecting the recorded date from damage or alteration. The first plunger is slidably disposed within the hollow second plunger, and the hollow second plunger is slidably disposed within the syringe barrel during use. A second syringe may be coupled to the system to provide syringe-to-syringe mixing.

### BACKGROUND OF THE INVENTION

1. The Field of the Invention

The present application is directed to devices for mixing, storing and dispensing time-sensitive dental compositions. More particularly, the application is directed to methods and systems for mixing, storing and dispensing two-part dental compositions that can become destabilized over time within their useful life.

2. The Relevant Technology

Many chemical formulations are packaged in two initially separate parts, often known as A and B components. Separate storage of the A and B components is often necessary where the composition resulting from mixing is unstable over time. For example, a self-etching dental primer composition may be provided in two initially separate parts to prevent the acid component from slowly destabilizing the polymerizable resin component by hydrolyzing off the functional group(s) to which the backbone of the resin is chemically bonded. Although such destabilization may not occur immediately upon mixing, with many such compositions, it is often recommended that the composition be used up or discarded within a certain time period (e.g., 30, 60, or 90 days) after initial mixing.

Because such compositions are typically only used in small amounts in any particular procedure, it can be difficult to determine how much time has elapsed since initial mixing, and whether or not the useful shelf-life of the mixed composition has expired. Simply discarding composition remaining after a single use is effective in preventing use of shelf-life expired product, but is wasteful and unnecessarily expensive. Alternatively, it may be possible to write the date of mixing onto the outside of the mixing or storage syringe. However, such writing can easily become smeared, smudged, altered, worn away, or otherwise rendered illegible, particularly when stored in a typical dental or medical environment. In light of the above, it would be an advantage to provide a time-indicating mixing system for use with a two-part composition that would allow a user to record a mixing date within the system for later reference that would be protected from damage or alteration.

### SUMMARY OF THE INVENTION

The present invention is directed to a time-indicating mixing, storing and dispensing syringe-in syringe system for a time-sensitive two-part dental composition. The system includes a first plunger, a hollow second plunger configured to contain a first component, and a syringe barrel configured to contain a second component. When assembled, the first plunger is slidably disposed within the hollow second plunger, and the hollow second plunger is slidably disposed within the syringe barrel. The system further includes means for indicating a recorded date when a first component was mixed with a second component and means for protecting the recorded date from damage or alteration.

An example of means for indicating a recorded date comprises an indented substantially flat label surface formed into a portion of the first plunger on which can be recorded a mixing date. The date may be written directly onto the label surface. Alternatively, the date may be written on an adhesive label which is afterwards adhered to the indented substantially flat label surface. An example of means for protecting the recorded date comprises forming at least a portion of the hollow second plunger so as to be transparent or translucent. Pressing the first plunger (including the label surface) into the hollow second plunger protects the recorded date, as the recorded date is covered and protected from damage or alteration by the hollow second plunger. The recorded date advantageously remains visible to the user through the transparent or translucent hollow second plunger.

The inventive syringe-in-syringe mixing system allows a user to mix a two-part dental composition while at the same time providing a record of the mixing date when the first and second components were mixed together. Advantageously, the recorded date is protected from damage or alteration. The system is particularly useful with two-part compositions that expire, lose potency or otherwise become unusable after a certain period of time has elapsed (e.g., a two-part self etching primer having a shelf-life of about thirty days after initial mixing).

Advantageously, the means for protecting the recorded date from damage or alteration may further include a locking mechanism to prevent the first plunger from being withdrawn once inserted into the hollow second plunger, so as to provide further protection against damage or alteration of the recorded date. For this purpose, the first plunger may advantageously comprise a plurality of longitudinally extending interlock ribs or an annular interlock ring formed near its proximal end. The interlock ribs or annular interlock ring are configured for insertion into the hollow second plunger, which includes a corresponding surface that engages the interlock ribs or ring so as to prevent the first plunger from being withdrawn once the ribs or ring have been inserted into the hollow second plunger, thereby locking the first plunger within the hollow second plunger.

The hollow second plunger advantageously includes a transparent or translucent window portion (or the whole hollow second plunger may be transparent or translucent), so as to allow a user to read the recorded date through the hollow second plunger. Because the first plunger is covered by the hollow second plunger, the recorded date is advantageously protected from damage or alteration that might otherwise occur if the recorded date were exposed (e.g., simply written on an outside surface of an exposed syringe barrel). Providing a locking mechanism for preventing withdrawal of the first plunger after insertion advantageously provides additional protection to the recorded date.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 is a perspective view of an exemplary syringe-in-syringe mixing system according to the present invention;

FIG. 2A is a perspective view of an exemplary first plunger of the inventive syringe-in-syringe mixing system including an exemplary locking mechanism;

FIG. 2B is a perspective view of an alternative first plunger of the inventive syringe-in-syringe mixing system including an alternative locking mechanism;

FIG. 3A is a perspective view of an exemplary hollow second plunger of the inventive syringe-in-syringe mixing system including a rupturable membrane at a distal end of the hollow second plunger;

FIG. 3B is a close up cross sectional view of a distal end of the hollow second plunger and rupturable membrane of FIG. 2A;

FIG. 3C is a perspective view of an alternative hollow second plunger of the inventive syringe-in-syringe mixing system including a valve at a distal end of the hollow second plunger;

FIG. 4A illustrates the syringe-in-syringe mixing system of FIG. 1 coupled to a second syringe barrel and associated plunger so as to form a syringe-to-syringe mixing system;

FIG. 4B illustrates a dental practitioner recording the mixing date onto the flat label surface of the first plunger of the mixing system of FIG. 4A prior to mixing the first and second components together;

FIG. 4C illustrates the first plunger being pressed into the hollow second plunger so as to cause the rupturable membrane of the hollow second plunger to break and the first component to mix with the second component;

FIG. 4D illustrates the first plunger in a locked configuration relative to the hollow second plunger;

FIGS. 5A-5B illustrates the exemplary mixing system of FIG. 4A with the user altermatingly pressing the plungers located at the distal and proximal ends of the system so as to cycle the commingled first and second components back and forth so as to form a homogeneous mixture; and

FIG. 6 illustrates the user dispensing a portion of the mixed two-part composition onto a pad for application to a desired surface.

### DETAILED DESCRIPTION OF THE PREFERRED EMBODIMENTS

I. Introduction

The present invention is directed to a time-indicating syringe-in-syringe mixing system for mixing, storing and dispensing a two-part composition. The system includes a first plunger, a hollow second plunger configured to contain a first component, a syringe barrel configured to contain a second component, means for indicating a recorded date when a first component was mixed with a second component, and means for protecting the recorded date from damage or alteration. When assembled, the first plunger is slidably disposed within the hollow second plunger, and the hollow second plunger is slidably disposed within the syringe barrel. The inventive mixing, storing and dispensing system advantageously allows a user to mix a two-part composition and to visibly record on the system for later reference the date as to when mixing occurred. The recorded date is advantageously protected from being damaged or altered. The visible recorded date indicates to the user how much shelf life remains until the mixed composition should be used up or discarded.

II. Exemplary Syringe-in-Syringe Mixing Systems

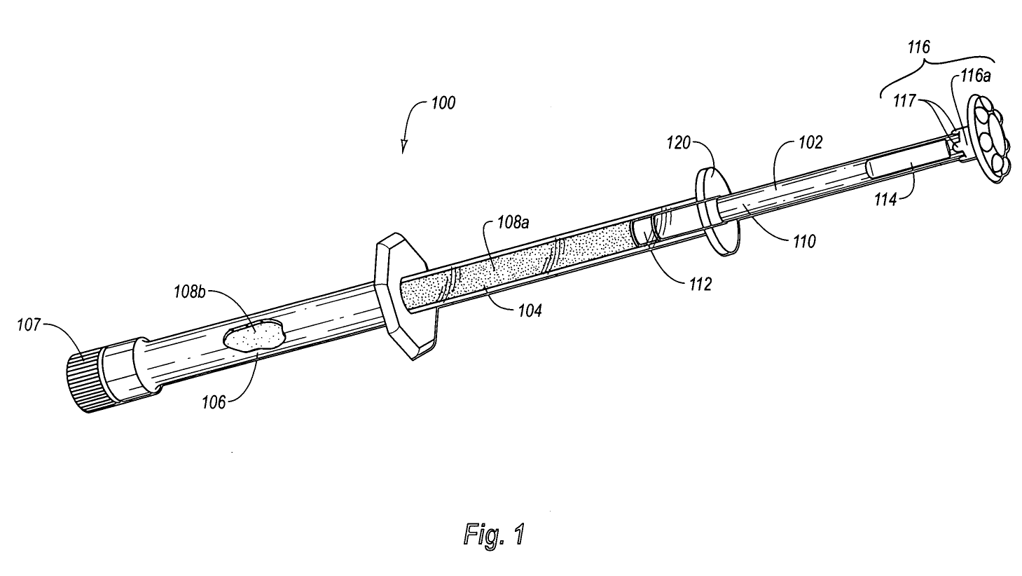

FIG. 1 illustrates an exemplary syringe-in-syringe mixing system 100 according to the present invention. System 100 includes a first plunger 102, a transparent or translucent hollow second plunger 104, and a syringe barrel 106 with a cap 107 at a distal end of syringe barrel 106. First plunger 102 is slidably disposed within hollow second plunger 104, which is slidably disposed within syringe barrel 106. As illustrated, hollow second plunger 104 is configured to contain a first component 108a, and syringe barrel 106 is configured to contain a second component 108b. First plunger 102 includes an elongate stem 110 and a sealing plug 112 at a distal end of stem 110. The sealing plug has a cross-sectional shape corresponding to a cross-sectional shape of the inside of hollow second plunger 104 (e.g., cylindrical). As shown, a substantially flat label surface 114 is formed into cylindrical elongate stem 110 near a proximal end of cylindrical elongate stem 110. The label surface 114 may advantageously be indented or recessed into the elongate stem 110.

First and second components 108a and 108b may each be a liquid, or one may be a solid powder, as dictated by the characteristics of the two-part composition to be mixed. One contemplated two-part composition is a two-part self etching dental primer composition described in U.S. patent application Ser. No. 11/261,171, filed Oct. 28, 2005, and entitled SELF-ETCHING DENTAL PRIMER COMPOSITIONS AND METHODS AND SYSTEMS UTILIZING SUCH COMPOSITIONS, herein incorporated by reference.

Depending on the length of hollow second plunger 104 and first plunger 102 relative to syringe barrel 106, locating label surface 114 near a proximal end of first plunger 102 allows a user to view the label surface 114 and a date recorded thereon even after first plunger 102 and hollow second plunger 104 have be fully inserted into syringe barrel 106 even if syringe barrel 106 is opaque. In other words, forming hollow second plunger 104 and first plunger 102 so as to be approximately equal in length and longer than syringe barrel 106 allows the proximal ends of hollow second plunger 104 and first plunger 102 to extend out of syringe barrel 106, even when fully inserted. For example, if syringe barrel 106 is shorter than hollow second plunger 104 and first plunger 106 by about the length of the label surface 114 (e.g., between about 0.75 inch and about 1 inch), label surface 114 and a date recorded thereon will still be visible to the user when the last of the composition is dispensed. Keeping label surface 114 and a date recorded thereon visible through hollow second plunger 104 is helpful to the user as it indicates whether the shelf life of the composition has expired. Alternatively, or in addition, the barrel 106 may itself be sufficiently transparent or translucent so that the recorded date remains visible even if partially or completely inserted into barrel 106.

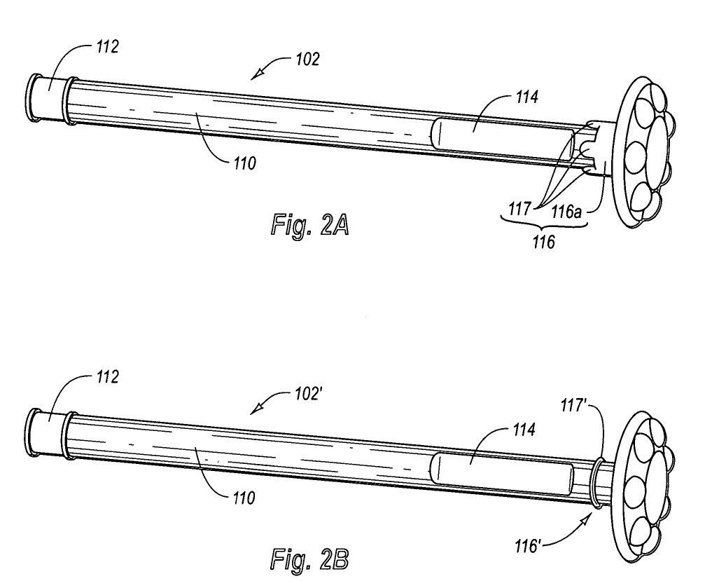

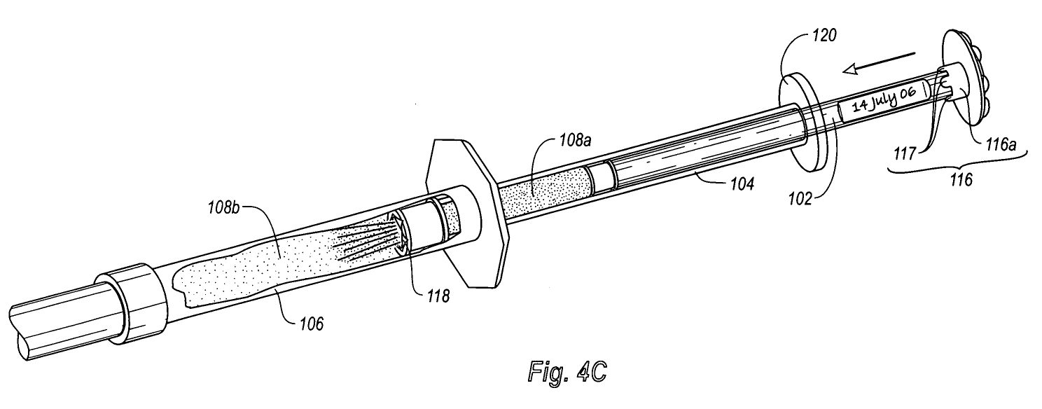

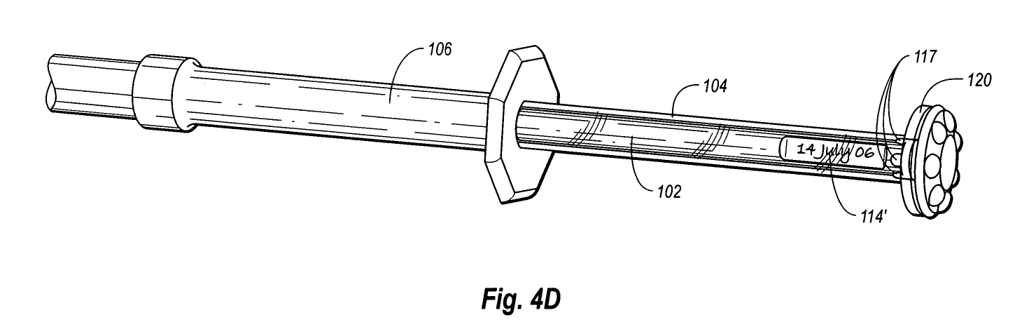

As perhaps best seen in FIG. 2A, a locking mechanism 116 may advantageously be included near a proximal end of first plunger 102 to prevent withdrawal of first plunger 102 from second plunger 104 once inserted. Such a locking mechanism further protects the recorded date from damage or alteration. Illustrated locking mechanism 116 comprises a circumferentially extending portion of enlarged diameter 116a (relative to the remainder of stem 110), with a plurality of longitudinally extending interlock ribs 117. In use, interlock ribs 117 are inserted into hollow second plunger 104, where the ribs 117 bias against the inside wall of hollow second plunger 104. The system is configured such that when first plunger 102 is fully inserted into hollow second plunger 104, circumferentially extending portion 116a rests within flange 120 of hollow second plunger 104, while interlock ribs 117 extend distally into hollow second plunger 104, past flange 120. Because flange 120 provides increased barrel strength relative to the remainder of hollow second plunger 104, little or no deformation occurs to the inside wall of hollow second plunger 104 on account of portion 116a, but deformation is caused by ribs 117, resulting in associated indentations being formed into the inside wall of hollow plunger 104 distal to flange 120, preventing, or at least inhibiting, later removal of first plunger 102 from hollow second plunger 104 (e.g., see FIG. 4D).

FIG. 2B illustrates an alternative first plunger 102′ including a cylindrical elongate stem 110, a sealing plug 112, and an indented substantially flat label surface 114. The principle difference between the first plunger 102′ and first plunger 102 of FIG. 2A and FIG. 1 is that first plunger 102′ includes an alternative locking mechanism 116′ comprising an annular interlock ring 117′ rather than the enlarged diameter portion 116a and plurality of interlock ribs 117 of the embodiment of FIG. 2A. Similar to interlock ribs 117, annular interlock ring 117′ causes the formation of an indentation or groove within the inside wall of hollow second plunger 104. Annular interlock ring 117′ resides in the formed groove, preventing, or at least inhibiting, pull out of first plunger 102 once fully inserted into hollow second plunger 104. Either of locking mechanisms 116 or 116′ further protects information (i.e., the mixing date) recorded on flat label surface 114 from altering by preventing pull out of first plunger 102.

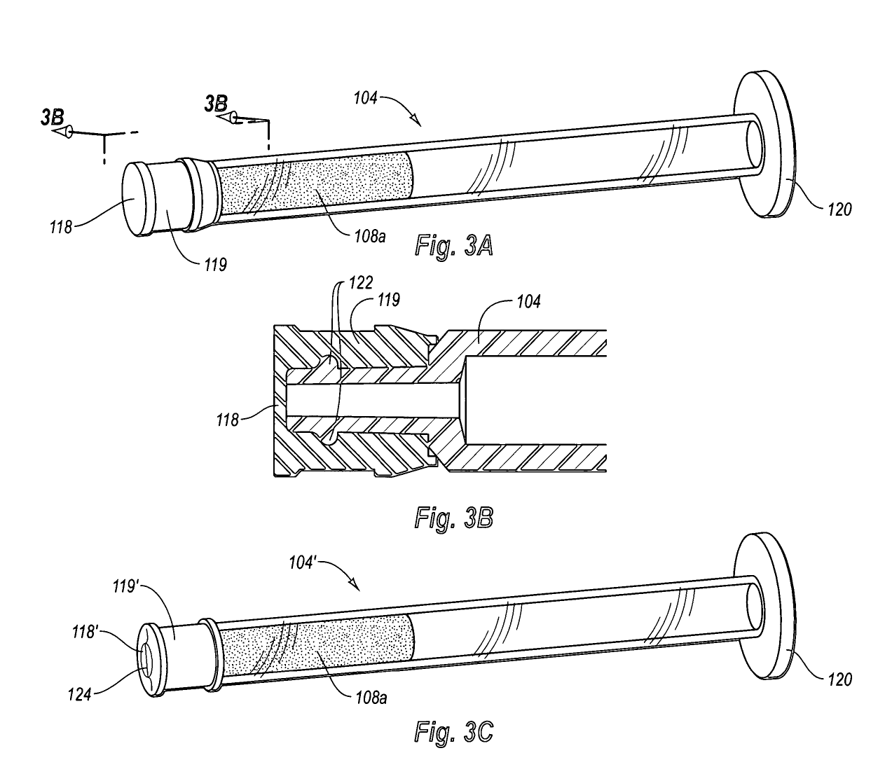

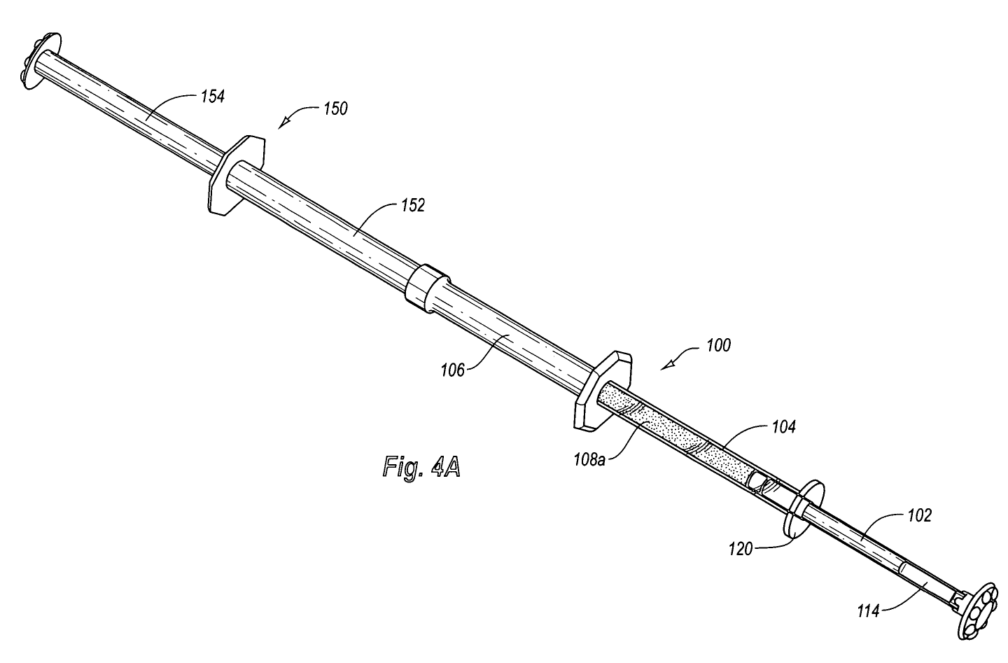

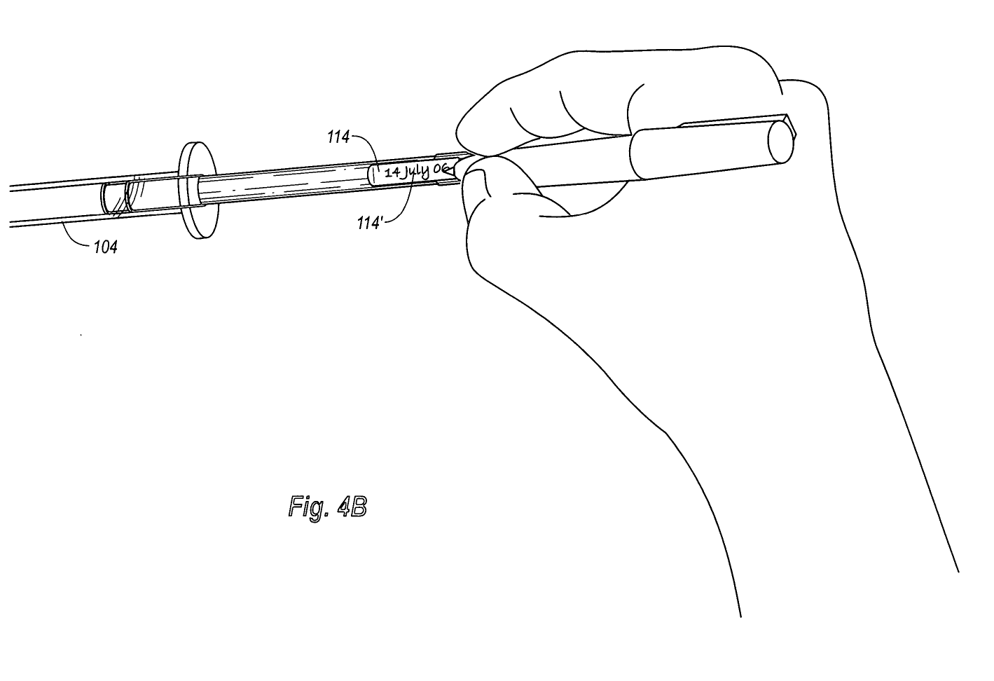

FIG. 3A is a perspective view of hollow second plunger 104 which includes a second sealing plug 119 having a rupturable membrane 118 at a distal end of hollow second plunger 104. Rupturable membrane 118 seals off the distal end of hollow second plunger 104, separating first component 108a from a second component 108b contained within the syringe barrel 106 (see FIG. 1) until the user intentionally ruptures membrane 118, causing first component 108a to be forced into syringe barrel 106, where the two components are mixed together. As seen in FIG. 3B, rupturable membrane 118 initially seals off a distal end of hollow second syringe 104. The distal portion of hollow second plunger 104 over which sealing plug 119 is fitted advantageously includes an enlarged annular ridge 122 that prevents plug 119 from being separated from hollow second plunger 104 during rupture of rupturable membrane 118.

Sealing plug 119 and rupturable membrane 118 may advantageously be formed of a thermoplastic elastomer (TPE), which advantageously provides an excellent seal against syringe barrel 106, while also providing a desired strength to rupturable membrane 118. Advantageously, the system may be configured so that the force required to rupture membrane 118 is approximately equal to the force required to insert and engage the locking mechanism (e.g., interlock ribs 117 or annular interlock ring 117′) of first plunger 102 into hollow second plunger 104. Such a configuration provides a smooth and continuous movement and feel during use of the system as first plunger 102 is pressed into hollow second plunger 104, rupturing membrane 118 and locking first plunger 102 into hollow second plunger 104. Rupturable membrane 118 preferably has a thickness ranging from about 0.0005 inch to about 0.04 inch, more preferably from about 0.002 inch to about 0.025 inch, and most preferably from about 0.005 inch to about 0.015 inch. Of course, the actual thickness of rupturable membrane 118 will depend on the strength and other physical properties of the selected material, along with the configuration and desired level of force required to break the membrane 118 and/or engage the selected locking mechanism. One particularly suitable material from which to form sealing plug 119 and rupturable membrane 118 is ENGAGE, a TPE sold by DuPont-Dow Elastomers located in Wilmington, Del.

FIG. 3C illustrates an alternative hollow second plunger 104′ including an alternative second sealing plug 119′. The principle difference between hollow second plunger 104′ of FIG. 3C and hollow second plunger 104 of FIG. 3B is that plug 119′ includes a pressure sensitive valve (e.g., a one way duck bill valve) 118′ rather than a rupturable membrane. Pressure sensitive valve 118′ acts to initially separate first component 108a within hollow second plunger 104′ from a second component 108b contained within syringe barrel 106 (see FIG. 1). Although pressure sensitive valve 118′ is illustrated as a duck bill one-way valve, any type of valve capable of initially separating the first and second components may alternatively be used. Pressure sensitive valve 118′ includes a slit 124 at a proximal end of plug 119′ through which the first component may be delivered for mixing with the second component.

III. Exemplary Method of Use

FIG. 4A illustrates an exemplary syringe-in-syringe mixing system 100 coupled to a second syringe 150 including a second syringe barrel 152 and an associated plunger 154. As seen in FIG. 4B, the user is able to record the mixing date 114′ on label surface 114 prior to fully pressing first plunger 102 into hollow second plunger 104. As illustrated, the user may write directly onto label surface 114 (e.g., with a pen or marker), or alternatively the date may be written on an adhesive label bearing an adhesive (e.g., a pressure sensitive adhesive) which may be subsequently adhered over the label surface 114. In either case, the recorded date 114′ is advantageously not located on a surface of stem 110 that will be rubbed against the inside wall of hollow second plunger 104, which may otherwise cause the date to be smeared, worn away, or otherwise rendered illegible.

Once the mixing date 114′ has been recorded on label surface 114, the user may press first plunger 102 into hollow second plunger 104 so as to compress first component 108a. As shown in. FIG. 4C, once a sufficient force is applied, rupturable membrane 118 breaks causing first component 108a to be expressed under pressure from hollow second plunger 104 into syringe barrel 106 where it mixes with second component 108b. As described above, the system may advantageously be configured such that a force required to rupture membrane 118 is approximately equal to a force required to insert and lock locking mechanism 116 (i.e., enlarged diameter portion 116a and interlocking ribs 117) of the cylindrical elongate stem 110 into hollow second plunger 104, although it is not required. FIG. 4D illustrates the system once first plunger 102 has been fully inserted into hollow second plunger 104. In this configuration, first plunger 102 is locked into hollow second plunger 104. As seen, recorded date 114′ is protected by and visible through transparent or translucent hollow second plunger 104.

In the locked configuration as shown, it is difficult, if not impossible, to withdraw first plunger 102 from hollow second plunger 104 without destroying the system. Enlarged diameter portion 116a is disposed within the center of flange 120, while ribs 117 extend distally from flange 120 further into hollow second plunger 104. Because flange 120 has increased barrel strength relative to the area of hollow second plunger 104 immediately distal to flange 120, the inside wall surface of hollow second plunger will be deformed by ribs 117 so as to form a depression into the portion of the inside wall contacted. At the same time, the inside surface of hollow second plunger 104 directly under flange 120 will be deformed only slightly if at all because of the increased barrel strength of the flange region 120 compared to the region contacted by ribs 117. In other words, ribs 117 create an interlock with the inside surface of hollow second plunger 104, preventing, or at least inhibiting, subsequent withdrawal of first plunger 102 from hollow second plunger 104.

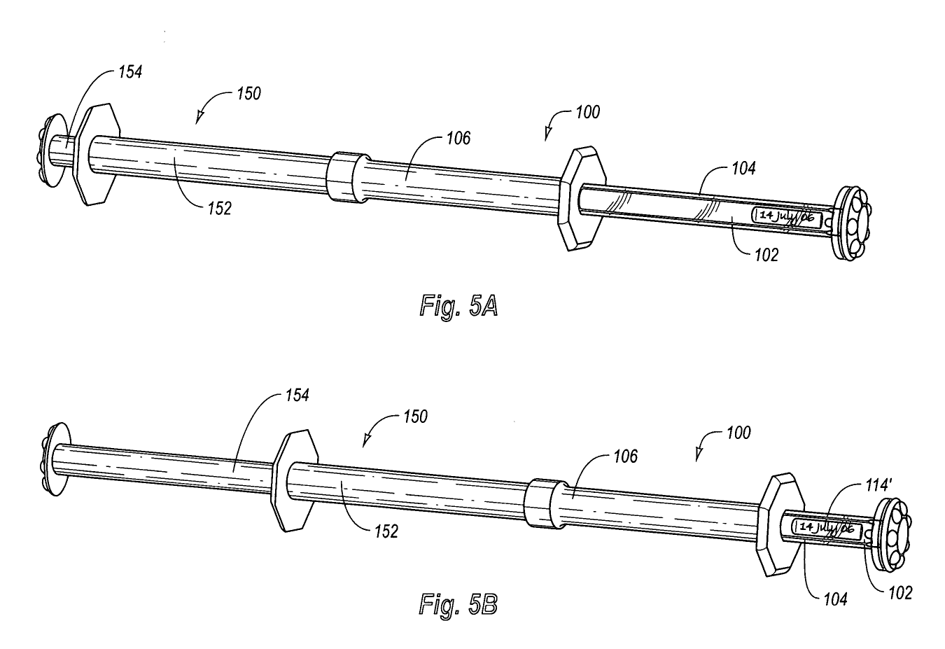

As seen in FIGS. 5A and 5B, the second syringe 150 may be used to more completely mix first components 108a and second component 108b. The user may altermatingly press hollow second plunger 104 and plunger 154 in order to cycle the two-part composition back and forth between barrel 106 and second syringe barrel 152, resulting in more homogeneous mixing. Although shown with a second syringe 150 coupled to system 100, it is to be understood that sufficient mixing may be achieved simply within the syringe-in-syringe mixing system 100, particularly where the rupturable membrane or valve is configured to only pass first component 108a for mixing with second component 108b under a pressure sufficiently high to cause jetting of the first component into the second component (e.g., so as to create turbulence sufficient to mix the two components together). In such a configuration, the distal end of syringe barrel 106 may simply be capped during mixing within the syringe-in-syringe mixing system 100 (see FIG. 1). The cap 107 may include a check-valve or other vent (not shown) that permits air within barrel 106 to be expelled as first component 108a is expressed into barrel 106. Any check-valve known in the art can be used or modified to attach to barrel 106.

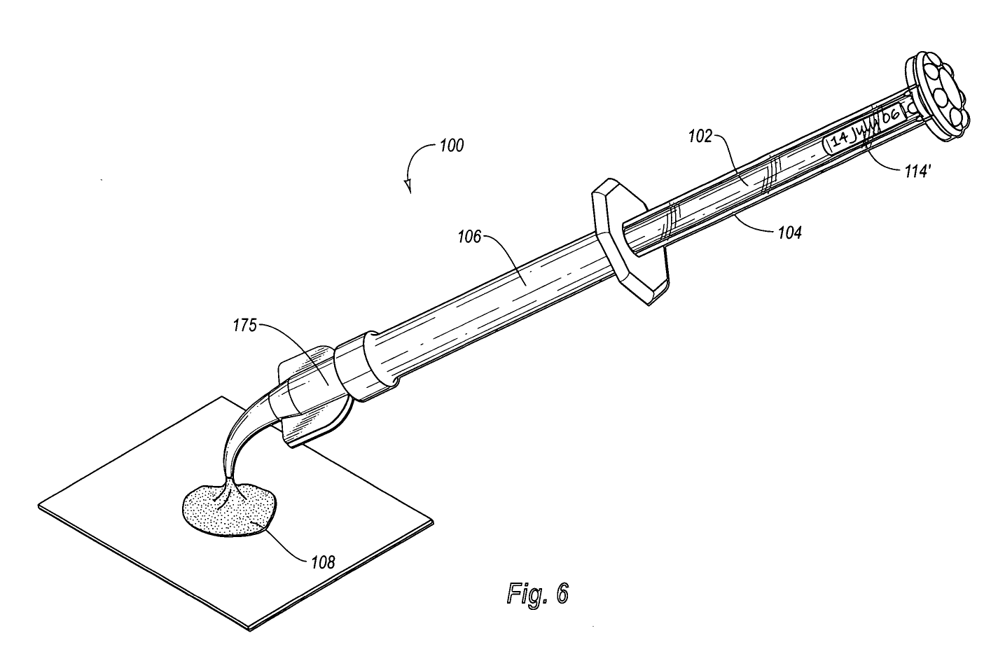

FIG. 6 illustrates the system 100 with a dispensing tip 175 coupled at a distal end of barrel 106 so as to allow the user to dispense the mixed two-part composition 108. In the case where a second syringe 150 is used to mix the components, the mixed composition is forced back into barrel 106, and second syringe 150 is detached from barrel before attaching dispensing tip 175. As illustrated, composition 108 maybe dispensed onto a pad for subsequent application (e.g., with a brush tool). Alternatively composition 108 may be dispensed directly onto a tooth or other surface, depending on the preference of the user.

It will be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A time-indicating mixing, storing and dispensing system for use with a two-part dental composition that, when mixed together, yield a time-sensitive dental composition, the system comprising:
a first plunger;
a hollow second plunger configured to contain therein a first component, the hollow second plunger being adapted to receive the first plunger therein in slidable engagement;
a syringe barrel configured to contain therein a second component, the syringe barrel being adapted to receive the hollow second plunger therein in slidable engagement;
means for indicating a recorded date when a first component initially contained within the hollow second plunger is mixed with a second component initially contained within the syringe barrel; and
means for protecting the recorded date from damage or alteration.

2. A time-indicating mixing, storing and dispensing system as recited in claim 1, wherein the means for indicating a recorded date is disposed on the first plunger.

3. A time-indicating mixing, storing and dispensing system as recited in claim 2, wherein the first plunger comprises an elongate stem and a sealing plug at a distal end of the elongate stem, the means for indicating a recorded date comprising an indented label surface formed into a portion of the elongate stem on which can be recorded a date.

4. A time-indicating mixing, storing and dispensing system as recited in claim 3, wherein the indented label surface is formed near a proximal end of the elongate stem.

5. A time-indicating mixing, storing and dispensing system as recited in claim 1, wherein the means for protecting the recorded date from damage or alteration comprises a transparent or translucent portion of the hollow second plunger that covers the recorded date once the first plunger is inserted into the hollow second plunger.

6. A time-indicating mixing, storing and dispensing system as recited in claim 5, further comprises a locking mechanism for preventing, or at least inhibiting, the first plunger from being withdrawn from the hollow second plunger once fully inserted into the hollow second plunger.

7. A time-indicating mixing, storing and dispensing system as recited in claim 6, wherein the locking mechanism comprises a plurality of interlock ribs formed near a proximal end of the first plunger, the plurality of interlock ribs being configured for insertion into the hollow second plunger so as to prevent the first plunger from being withdrawn once the plurality of interlock ribs have been, inserted into the hollow second plunger.

8. A time-indicating mixing, storing and dispensing system as recited in claim 6, wherein the locking mechanism comprises an annular interlock ring formed near a proximal end of the first plunger, the annular interlock ring being configured for insertion into the hollow second plunger so as to prevent the first plunger from being withdrawn once the annular interlock ring has been inserted into the hollow second plunger.

9. A time-indicating mixing, storing and dispensing system as recited in claim 1, further comprising a second syringe barrel coupleable to a distal end of the syringe barrel so as to allow syringe-to-syringe mixing of a first component with a second component.

10. A time-indicating mixing, storing and dispensing system as recited in claim 1, further comprising separation means for initially separating a first component within the hollow second plunger from a second component within the syringe barrel.

11. A time-indicating mixing, storing and dispensing system as recited in claim 10, wherein the separation means comprises a rupturable membrane.

12. A time-indicating mixing, storing and dispensing system as recited in claim 11, wherein the rupturable membrane comprises a thermoplastic elastomer.

13. A time-indicating mixing, storing and dispensing system as recited in claim 10, wherein the separation means comprises a valve.

14. A time-indicating mixing, storing and dispensing system for use with a two-part dental composition that, when mixed together, yield a time-sensitive dental composition, the system comprising:
a first plunger comprising an elongate stem and a sealing plug at a distal end of the elongate stem;
a hollow second plunger configured to contain therein a first component, the first plunger being slidably disposed within the hollow second plunger during use, the second plunger including a transparent or translucent window portion;
a syringe barrel configured to contain therein a second component, the hollow second plunger being slidably disposed within the syringe barrel during use; and
an indented label surface formed into a portion of the elongate stem on which can be recorded a date when a first component initially contained within the hollow second plunger is mixed with a second component initially contained within the syringe barrel,
wherein a date recorded on the label surface can be viewed through the transparent or translucent window portion of the hollow second plunger once the first plunger is fully inserted into the hollow second plunger.

15. A time-indicating mixing, storing and dispensing system as recited in claim 14, further comprising a locking mechanism formed near a proximal end of the cylindrical elongate stem, the locking mechanism being configured for insertion into the hollow second plunger so as to prevent the first plunger from being withdrawn once the locking mechanism has been fully inserted into the hollow second plunger.

16. A time-indicating mixing, storing and dispensing system as recited in claim 15, further comprising a rupturable membrane for separating a first component within the hollow second plunger from a second component within the syringe barrel.

17. A time-indicating mixing, storing and dispensing system as recited in claim 16, wherein a force required to rupture the rupturable membrane is approximately equal to a force required to insert the locking mechanism of the cylindrical elongate stem into the hollow second plunger.

18. A method of mixing, storing and dispensing a time-sensitive composition formed by mixing together a two-part dental composition, the method comprising:
providing a time-indicating mixing, storing and dispensing system as recited in claim 14;
recording a date when a first component initially contained within the hollow second plunger is mixed with a second component initially contained within the syringe barrel on the label surface of the elongate stem; and
pressing the elongate stem of the first plunger into the hollow second plunger so as to cause the first component within the hollow second plunger to be mixed with the second component within the syringe barrel.

19. A method as recited in claim 18, wherein the date when the first component is mixed with the second component is written directly onto the indented label surface of the elongate stem.

20. A method as recited in claim 18, wherein the date when the first component is mixed with the second component is written on an adhesive label, the method further comprising adhering the adhesive label to the indented label surface of the elongate stem.

21. A method as recited in claim 18, further comprising:
inserting a locking mechanism on the elongate stem into the hollow second plunger so as to lock the first plunger within the hollow second plunger.
